(19) United States
(12) Patent Application Publication
(10) Pub. No.: US 2025/0107770 A1
(43) Pub. Date: Apr. 3, 2025

(54) SYSTEMS AND METHODS FOR NONCONTACT MONITOR OF CARDIAC ACTIVITIES

(71) Applicant: Toyota Motor Engineering & Manufacturing North America, Inc., Plano, TX (US)

(72) Inventors: Paul D. Schmalenberg, Ann Arbor, MI (US); Kleanthis Avramidis, Los Angeles, CA (US); Frederico Marcolino Quintao Severgnini, Ann Arbor, MI (US); Bryan Pardo, Evanston, IL (US)

(73) Assignee: Toyota Motor Engineering & Manufacturing North America, Inc., Plano, TX (US)

(21) Appl. No.: 18/374,964

(22) Filed: Sep. 29, 2023

(51) Int. Cl. A61B 7/00; A61B 5/00; A61B 5/319; H04R 3/04

(52) U.S. Cl. CPC A61B 7/00; A61B 5/319; A61B 5/7225; A61B 5/7267; H04R 3/04; A61B 2562/0204; H04R 2430/01

(57) ABSTRACT

Systems and methods for contactless monitoring of cardiac activities include a vibration sensor operable to collect audio input signals of a user and a processor. The processor is operable to extract, using an audio model, cardiac sound data from the audio input signals, and transfer, using a trained neural network, the cardiac sound data into the simulated electrocardiogram (ECG) data based on a weak alignment between cardiac sounds and ECG.

### TECHNICAL FIELD

The present disclosure relates to cardiac monitoring device, and more particularly, to cardiac monitoring device using noncontact sensors.

### BACKGROUND

Health monitoring devices frequently require the use of components such as wires and electrodes attached to the user. This invasive method can lead to user discomfort, reduced mobility, potential risks of skin irritation and infection, and sleep disturbances. These factors can have a significant impact on the user's overall experience, data reliability, and monitoring convenience. Consequently, there is a demand for contactless health monitoring solutions.

### SUMMARY

In a first aspect, a system for contactless monitoring of cardiac activities includes a vibration sensor operable to collect audio input signals of a user and a processor. The processor is operable to extract, using an audio model, cardiac sound data from the audio input signals, and transfer, using a trained neural network, the cardiac sound data into the simulated electrocardiogram (ECG) data based on a weak alignment between cardiac sounds and ECG.

In a second aspect, a method for contactless monitoring of cardiac activities comprises extracting, using an audio model, cardiac sound data from audio input signals of a user collected using a vibration sensor, and transferring, using a trained neural network, the cardiac sound data into a ECG data based on a weak alignment between cardiac sounds and ECG.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a contactless cardiac monitoring system of the present disclosure, according to one or more embodiments shown and described herein;

FIG. 2 schematically depicts non-limiting components of the contactless cardiac monitoring system of the present disclosure, according to one or more embodiments shown and described herein;

FIG. 3A schematically depicts a raw collected cardiac audio waveform and a corresponding electrocardiogram (ECG) waveform of the present disclosure, according to one or more embodiments shown and described herein;

FIG. 3B schematically depicts a simulated ECG waveform generated using the current disclosed systems and methods, compared with a measured ECG waveform, according to one or more embodiments shown and described herein;

FIG. 4A schematically depicts a block diagram of using the operation of the contactless cardiac monitoring system to generate the simulated ECG signals based on the collected cardiac audio signals, according to one or more embodiments shown and described herein;

FIG. 4B schematically depicts a block diagram of training the contactless cardiac monitoring system using cardiac audio signals and ECG signals in associated with the cardiac audio signals, according to one or more embodiments shown and described herein;

FIG. 5 illustrates a flow diagram of illustrative steps for generating simulated ECG data using the contactless cardiac monitoring system of the present disclosure, according to one or more embodiments shown and described herein; and

FIG. 6 illustrates a flow diagram of illustrative steps for generating simulated ECG data using a neural network of the contactless cardiac monitoring system of the present disclosure, according to one or more embodiments shown and described herein.

### DETAILED DESCRIPTION

The present disclosure involves contactless cardiac monitoring systems and methods for a non-invasive monitor of a user's cardiac activities. Unlike the invasive monitoring device using electrodes placed on the skin of a user near the heart of the user, the embodiments of the present disclosure use audio sensors or acceleration sensors around the user, without any component physically contacting the user.

The contactless cardiac monitoring systems described herein offer desirable benefits over contact-based systems. One of the notable benefits is improved comfort and convenience for the users. By eliminating the need for electrodes or sensors directly attached to the user's skin, contactless monitoring reduces discomfort and allows individuals to maintain their daily routines without the constraints of wires and adhesive patches. This enhanced user experience can promote better compliance with monitoring protocols, as it minimizes disruptions to daily life and sleep patterns.

Furthermore, contactless systems mitigate the risk of skin irritation and infection, which can be concerns with contact-based monitoring that necessitates direct skin contact. Without the requirement for electrodes, users are less susceptible to skin-related issues, and the absence of physical contact lowers the potential for infection. Contactless monitoring also offers continuous monitoring capabilities and extended monitoring periods, making it suitable for both short-term and long-term monitoring requirements. Additionally, the ability to remotely monitor users using contactless systems enhances healthcare accessibility, particularly benefiting individuals in remote areas or with limited mobility, while also reducing the strain on healthcare facilities. Further, the contactless cardiac monitoring systems described herein offer the advantage of collecting cardiac signals without requiring the user to remain stationary. This is in contrast to certain systems that may necessitate targeting components to detect the user's position for signal collection.

Throughout the disclosure, contactless monitoring refers to monitoring a user without direct physical contact. For example, contactless monitoring is found when an accelerometer sensor is embedded in a chair in contact with a user's clothing when the user sits on the chair. The vibration sensor refers to a device that detects the vibration of matters, such as air, liquid, and solids. The vibration sensors may include, without limitations, audio sensors and accelerometer sensors. The audio sensor refers to a device that detects sound waves and converts the sound waves into electrical signals in a surrounding area.

Various embodiments of the methods and systems for clinical procedure training are described in more detail herein. Whenever possible, the same reference numerals will be used throughout the drawings to refer to the same or like parts.

As used herein, the singular forms “a,” “an” and “the” include plural referents unless the context clearly dictates otherwise. Thus, for example, reference to “a” component includes aspects having two or more such components unless the context clearly indicates otherwise.

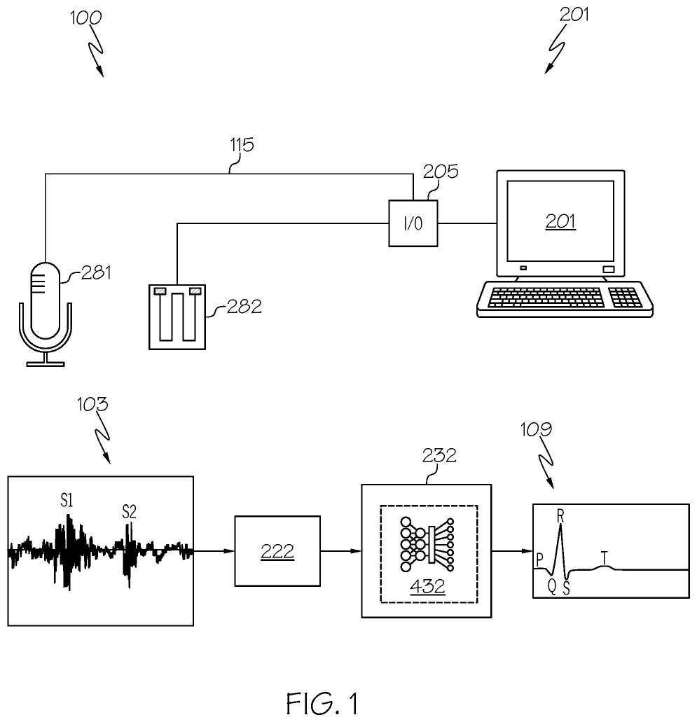

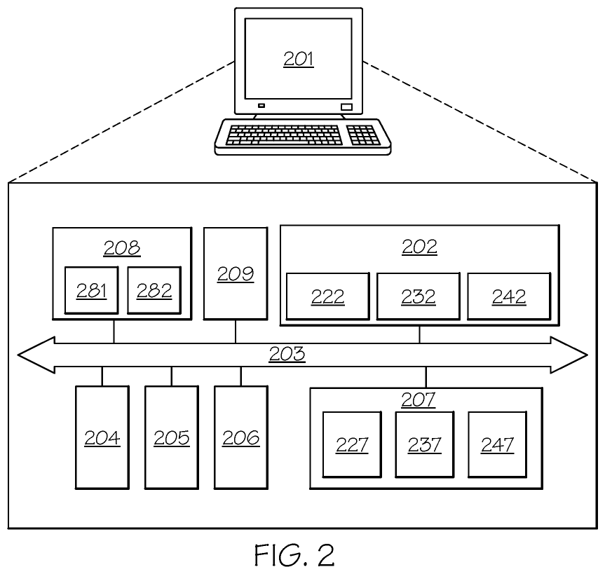

Turning to the figures, FIG. 1 schematically depicts an example contactless cardiac monitoring system 100. The example contactless cardiac monitoring system 100 includes a vibration sensor 208 (e.g. as illustrated in FIG. 2), such as, without limitations, an audio sensor 281 and an accelerometer sensor 282 operable to collect audio input signals of a user and a controller 201. The controller 201 may have a user interface 209, an input/output (I/O) hardware 205, and connections 115. The connections 115 connects components of the contactless cardiac monitoring system 100 and allows signal transmission between the components of the contactless cardiac monitoring system 100. For example, the connections 115 may connect the vibration sensors 208 such as the audio sensor 281 and the accelerometer sensor 282 to the controller 201 at the I/O hardware 205. The connections 215 may be formed from any medium that is capable of transmitting a signal such as, for example, conductive wires, conductive traces, optical waveguides, or the like. In some embodiments, the connections 215 may facilitate the transmission of wireless signals, such as WiFi, Bluetooth®, Near Field Communication (NFC), and the like. The user interface 104 may be, without limitation, a monitor, a display, a touchscreen, or a web-based interface. The user interface 209 may display a simulated electrocardiogram (ECG) data as an output.

The vibration sensor 208 may detect the mechanical vibrations of matter, including air, liquid, and solids, and generate audio input signals 103 to transmit to the controller 201. The vibration sensor 208 may be the audio sensor 281. The audio sensor 281 may be an air-coupled audio sensor, a condenser microphone, an electret microphone, or a piezoelectric microphone. The air-coupled audio sensor may use changes in air pressure to capture sound waves. The condenser microphone (or a capacitor microphone) may use a diaphragm and a backplate separated by a small air gap to capture sound.

The accelerometer sensor 282 may collect velocity and displacement information as a function of time and be further integrated into the cardiac sound data of the user. The accelerometer sensor 282 may be embedded in fixtures, such as, without limitations, a seat, a bed, a recline, or a sofa, such that, when the user sits on or leans on the fixture, the accelerometer sensor 282 may indirectly contact with the user to collect the vibrations of the user produced by the heart contraction. The accelerometer sensor 282 may be strategically positioned in the fixture to enhance the sensitivity to the vibrations generated by the user's heart contractions.

In some embodiments, the vibration sensors 208 may have the capability to gather omnidirectional vibrations, capturing sound equally from all directions without a specific emphasis on any particular source or direction. In other embodiments, the vibration sensors 208 can gather vibrations from specific directions to mitigate background noise. The choice between the omnidirectional and selective direction approaches may depend on the spatial design and user behavior within the environment. For instance, the vibration sensors 208 may be installed in a vehicle. When one or more users enter the vehicle, the vibration sensors 208 collect all vibrations, including airborne and body-transmitted audio vibrations, as well as those from vehicle components. The omnidirectional approach may be employed when the vibration sensors 208 are designed to capture all audio input signals 103 within the vehicle, regardless of the user's position. On the other hand, the selective direction approach may be employed when the vibration sensors 208 are designed to target and concentrate on a specific user, such as a driver who remains seated for an extended duration.

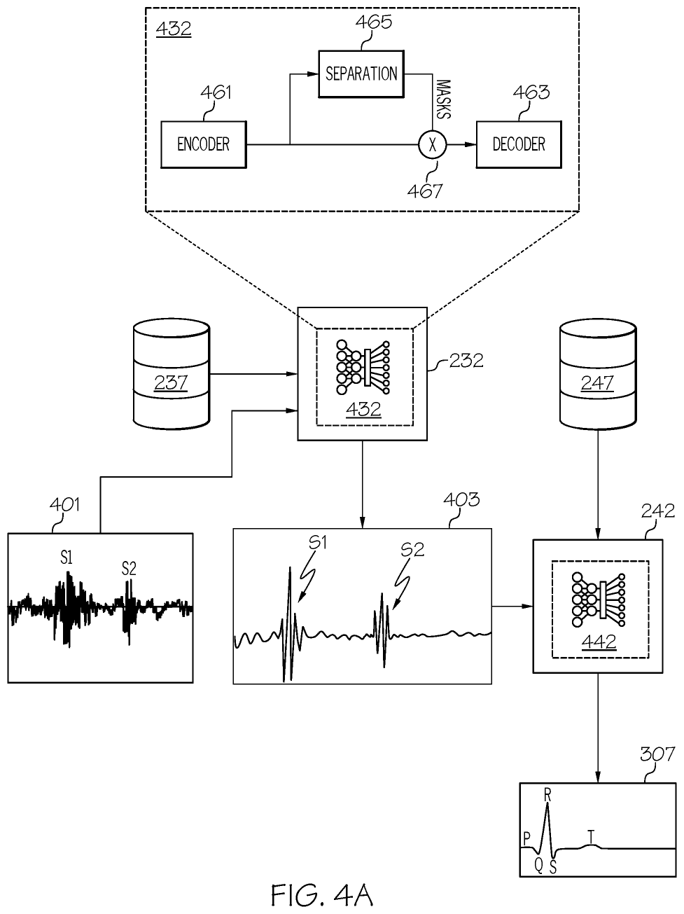

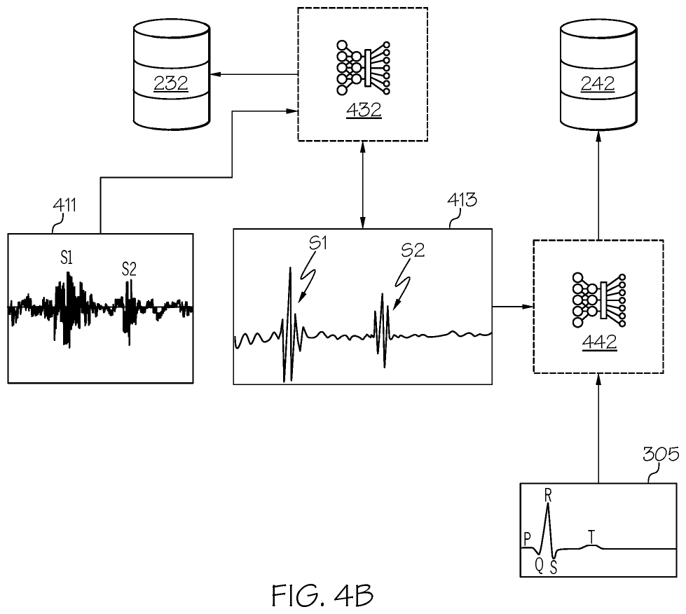

The collected audio signals 103 may be transmitted to the controller 201 via the connections 115 through the input/output hardware 205. The controller 201 includes a preprocessing module 222, a denoising module 232, and a weak alignment module 242 (e.g. as illustrated in FIG. 2). The denoising module 232 may include a first neural network 432 and the weak alignment module 242 may include a second neural network 442 (e.g. as illustrated in FIGS. 4A and 4B). In some embodiments, the system 100 may combine the denoising module 232 and the weak alignment module 242 together and include a single neural network such as the first neural network 432 for the combined module. The controller 201 may extract cardiac sound data 401 (e.g. as illustrated in FIG. 4) from the audio input signals 103 collected from the vibration sensors 208 using the preprocessing module 222. The controller 201 may further transfer the cardiac sound data 401 into a simulated electrocardiogram (ECG) data 109 based on a weak alignment between cardiac sounds and the ECG, using the denoising module 232 and the weak alignment module 242.

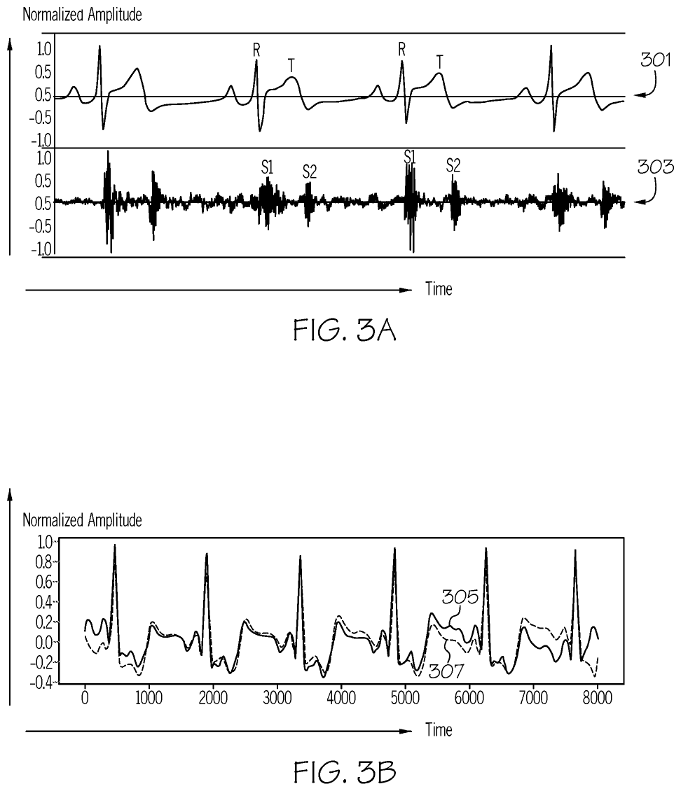

In embodiments, the preprocessing module 222 may filter, normalize, or segment the audio input signals 103. The segmentation of the audio input signal 103 may be cut into segments of audio input signals 103 of a few seconds to a few minutes to a few hours. Each segment of the audio input signals 103 may include more than one heartbeat cardiac audio signals. In some embodiments, the segments may include four heartbeats, such as illustrated in FIG. 3A. The system 100 may include one or more filter to isolate the audio input signals 103 to retain the cardiac audio signals 401. For example, the preprocessing module 222 may filter the audio input signals 103 to retain frequencies of the audio input signals 103 between 25 Hz and 50 Hz. In some other embodiments, the filtering procedure may choose a range of the retain frequencies of the audio input signals 103 determined based on the parameters of the system 100. For example, the selection of the range of the retain frequencies of the audio input signals 103 may be determined based on the preprocessing matching data 227 (e.g. as illustrated in FIG. 2), which may include, without limitations, parameters of the vibration sensors 208 (such as, without limitation, bandwidth and quality factors) and the processing signal band of the denoising module 232.

Further, the preprocessing module 222 may normalize the audio input signals 103 by adjusting amplitudes of the audio input signals 103 using peak amplitude normalization, root mean square normalization, or loudness normalization. The system 100 may compare with the preprocessing matching data 227 in determining the filtering, normalization, or segmentation to provide a desired preprocess to the audio input signal 103. After the audio input signals 103 are preprocessed, the system 100 may transfer the cardiac sound data 401 and output the simulated ECG data 109, as described in detail further below.

Referring to FIG. 2, example non-limiting components of the contactless cardiac monitoring system 100 are depicted. The contactless cardiac monitoring system 100 may include the controller 201. The controller 201 may include various modules. For example, the controller 201 may include the preprocessing module 222, the denoising module 232, and the weak alignment module 242. The controller 201 may further comprise various components, such as a memory component 202, a processor 204, an input/output hardware 205, a network interface hardware 206, a data storage component 207, and a local interface 203. The controller 201 may include one or more vibration sensors 208 and a user interface 209.

The controller 201 may be any device or combination of components comprising a processor 204 and a memory component 202, such as a non-transitory computer readable memory. The processor 204 may be any device capable of executing the machine-readable instruction set stored in the non-transitory computer readable memory. Accordingly, the processor 204 may be an electric controller, an integrated circuit, a microchip, a computer, or any other computing device. The processor 204 may include any processing component(s) configured to receive and execute programming instructions (such as from the data storage component 207 and/or the memory component 202). The instructions may be in the form of a machine-readable instruction set stored in the data storage component 207 and/or the memory component 202. The processor 204 is communicatively coupled to the other components of the controller 201 by the local interface 203. Accordingly, the local interface 203 may communicatively couple any number of processors 204 with one another, and allow the components coupled to the local interface 203 to operate in a distributed computing environment. The local interface 203 may be implemented as a bus or other interface to facilitate communication among the components of the controller 201. In some embodiments, each of the components may operate as a node that may send and/or receive data. While the embodiment depicted in FIG. 2 includes a single processor 204, other embodiments may include more than one processor 204.

The memory component 202 (e.g., a non-transitory computer-readable memory component) may comprise RAM, ROM, flash memories, hard drives, or any non-transitory memory device capable of storing machine-readable instructions such that the machine-readable instructions can be accessed and executed by the processor 204. The machine-readable instruction set may comprise logic or algorithm(s) written in any programming language of any generation (e.g., 1GL, 2GL, 3GL, 4GL, or 5GL) such as, for example, machine language that may be directly executed by the processor 204, or assembly language, object-oriented programming (OOP), scripting languages, microcode, etc., that may be compiled or assembled into machine readable instructions and stored in the memory component 202. Alternatively, the machine-readable instruction set may be written in a hardware description language (HDL), such as logic implemented via either a field-programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components. For example, the memory component 202 may be a machine-readable memory (which may also be referred to as a non-transitory processor-readable memory or medium) that stores instructions that, when executed by the processor 204, causes the processor 204 to perform a method or control scheme as described herein.

While the embodiment depicted in FIG. 2 includes a single non-transitory computer-readable memory component, other embodiments may include more than one memory module. The memory may be used to store the preprocessing module 222, the denoising module 232, and the weak alignment module 242. Each of the preprocessing module 222, the denoising module 232, and the weak alignment module 242 during operating may be in the form of operating systems, application program modules, and other program modules. Such program modules may include, but are not limited to, routines, subroutines, programs, objects, components, and data structures for performing specific tasks or executing specific abstract data types according to the present disclosure as will be described below.

The input/output hardware 205 may include a monitor, keyboard, mouse, printer, camera, microphone, speaker, and/or other device for receiving, sending, and/or presenting data. The network interface hardware 206 may include any wired or wireless networking hardware, such as a modem, LAN port, Wi-Fi card, WiMax card, mobile communications hardware, and/or other hardware for communicating with other networks and/or devices.

The data storage component 207 stores preprocessing matching data 227, historical cardiac sound data 237, historical ECG data 247, collected data generated by the vibration sensors 208, and operating data of the vibration sensors 208. The preprocessing module 222, the denoising module 232, and the weak alignment module 242 may also be stored in the data storage component 207 during operating or after operation.

Each of the preprocessing module 222, the denoising module 232, and the weak alignment module 242 may include one or more machine learning algorithms or neural networks, such as the first neural network 432 and the second neural network 442 (e.g. as illustrated in FIGS. 4A and 4B). The preprocessing module 222, the denoising module 232, and the weak alignment module 242 may be trained and provided machine learning capabilities via a neural network as described herein. By way of example, and not as a limitation, the neural network may utilize one or more artificial neural networks (ANNs). In ANNs, connections between nodes may form a directed acyclic graph (DAG). ANNs may include node inputs, one or more hidden activation layers, and node outputs, and may be utilized with activation functions in the one or more hidden activation layers such as a linear function, a step function, logistic (sigmoid) function, a tanh function, a rectified linear unit (ReLu) function, or combinations thereof. ANNs are trained by applying such activation functions to training data sets to determine an optimized solution from adjustable weights and biases applied to nodes within the hidden activation layers to generate one or more outputs as the optimized solution with a minimized error. In machine learning applications, new inputs may be provided (such as the generated one or more outputs) to the ANN model as training data to continue to improve accuracy and minimize error of the ANN model. The one or more ANN models may utilize one-to-one, one-to-many, many-to-one, and/or many-to-many (e.g., sequence to sequence) sequence modeling. The one or more ANN models may employ a combination of artificial intelligence techniques, such as, but not limited to, Deep Learning, Random Forest Classifiers, Feature extraction from audio, images, clustering algorithms, or combinations thereof. In some embodiments, a convolutional neural network (CNN) may be utilized. For example, a convolutional neural network (CNN) may be used as an ANN that, in a field of machine learning, for example, is a class of deep, feed-forward ANNs applied for audio analysis of the recordings. CNNs may be shift or space invariant and utilize shared-weight architecture and translation. Further, each of the various modules may include one or more generative artificial intelligence algorithms. The generative artificial intelligence algorithm may include a general adversarial network (GAN) that has two networks, a generator model and a discriminator model. The generative artificial intelligence algorithm may also be based on variation autoencoder (VAE) or transformer-based models.

Referring to FIGS. 3A and 3B, example cardiac audio waveforms and the ECG waveforms are illustrated. A correlated relationship is shown in a cardiac audio waveform 301 and a corresponding ECG waveform 303 for a period of four heartbeats. The cardiac audio waveform 301 exhibits a backdrop of background noise, intertwined with two distinct cardiac sounds accompanying each heartbeat. The initial heart sound, designated as S1, materializes through vibrations associated with the closure of the tricuspid and mitral valves at the onset of systole. S1 resonates for approximately 14 milliseconds, encompassing frequencies reaching up to roughly 500 Hz. Conversely, the subsequent heart sound, referred to as S2, is generally linked to vibrations generated by the closure of the aortic and pulmonary valves at the culmination of systole. Although S2's duration typically pales in comparison to that of S1, it possesses a broader spectral bandwidth.

Simultaneously, the ECG waveform 303 portrays the electrical activity coursing through a user's heart. As depicted in FIGS. 1 and 3, the ECG waveform 303 or the simulated ECG data 109 charts the atrial muscle fiber depolarization as the “P” wave, followed by the collective depolarization of ventricular muscle fibers represented by the “Q,” “R,” and “S” waves. The segment of the ECG waveform 303 or the simulated ECG data 109 signifying the repolarization of ventricular muscle fibers is denoted as the “T” wave. During the intervals between heartbeats, the ECG waveform 303 or the simulated ECG data 109 reverts to an isopotential baseline. Accordingly, four peaks of the ECG waveform 303 or the simulated ECG data 109, including the P peak, Q peak, R peak, S peak, and a T peak, are associated features in one heartbeat.

Comparing the cardiac audio waveform 301 to the ECG waveform 303, a weak alignment is found between the cardiac sounds and the ECG based on correlated pair features between the cardiac sounds and the ECG. The correlated pair features include the R peaks of the ECG waveform 303 corresponding to the S1 features of the cardiac audio waveform 301, and the T peaks in the ECG waveform 303 corresponding to the S2 features of the cardiac sounds. The current systems and methods transfer the collected cardiac sound data into simulated ECG data 109 based on the correlation pair features between the cardiac sound and the ECG.

The trained second neural network 442 may generate the simulated ECG waveform 307 based on the measured cardiac sound signals. For example, as illustrated in FIG. 3B, The simulated ECG waveform 307 is illustrated against the measured ECG waveform 305 corresponding to the measure cardiac sound signals, where the simulated ECG waveform 307 matches the measured ECG waveform 305 in terms of the peak location and waveform shapes. In some embodiments, a mean peak error of R peaks and T peaks between the measured ECG waveform 305 and the simulated ECG waveform 307 is less than 5 ms in 70% of the samples. The peak intervals, such as RR intervals, may have a mean peak interval error of 7 ms. An estimated heart rate may have a mean heart rate error of 0.5 BPM.

FIGS. 4A and 4B schematically depict the block diagrams of the methods for using the operation of the contactless cardiac monitoring system 100. The controller 201 includes the denoising module 232 and the weak alignment module 242. The denoising module 232 may include the first neural network 432 and the weak alignment module 242 may include the second neural network 442. In some embodiments, the denoising module 232 and the weak alignment module 242 may be combined into a single module including a single neural network fulfilling both the denoising function and the simulation function. The controller 201 may extract cardiac sound data 401 from the audio input signals 103 (as illustrated in FIG. 1) collected from the vibration sensors 208 using the preprocessing module 222. The controller 201 may further transfer the cardiac sound data 401 into a simulated electrocardiogram (ECG) data 109 based on a weak alignment between cardiac sounds and the ECG, using the denoising module 232 and the weak alignment module 242.

FIG. 4A depicts an example method to generate the simulated ECG signals 307 based on the cardiac audio signals 401. The denoising module 232 may analyze the cardiac sound data 401 to determine if a noise level of the cardiac sound data 401 satisfies the requirements provided in the contactless cardiac monitoring system 100. In embodiments, the denoising module 232 may include one or more tolerance thresholds and apply a denosing model through the trained first neural network 432 in comparing the historical cardiac sound data 237 whether the noise level of the cardiac sound data 401 satisfies the requirements. In response to the determination that the noise level is beyond an accepted level, the denoising module 232 may apply the trained first neural network 432 in removing the noises in the cardiac sound data 401 and extracting denoised cardiac sound signals 403.

In embodiments, the first neural network 432 may include a sequence-to-sequence learning algorithm to separate the noises in the cardiac sound signals 401. As illustrated in FIG. 4A, the first neural network 432 may include an encoder 461, a separator 465 including one or more hidden layers of separation, and a decoder 463. The first neural network 432 may feed the cardiac sound data 401 into the encoder 461 to generate a lower-dimensional representation of the cardiac waveform. The lower-dimensional representation may include a number of features determined based on the channels in the cardiac sound signals 401 (e.g. the source of sounds, the number and types of the vibration sensors 208) and the residual path of the subsequent convolutional blocks. The separator 465 may then generate one or more masks indicative parts of the cardiac sound data out of the background noises. The first neural network may apply the estimated masks to cardiac sound signals 401 to isolate the cardiac sound data 401 in a masking process 467 and reconstruct, using the decoder 463, the isolated cardiac sound data to generate the denoised cardiac sound signals 403.

In some embodiments, the encoder 461 may conjunct with a layer normalization operation and an activation function operation. The encoded cardiac sound signals may be normalized and weighted through the activation function before fed to the separator 465. The separator 465 may generate a representation of the cardiac sound signals for each user when one or more users are present in the space where the vibration sensors 208 may collect the environment sounds. The separator 465 then create multiple of masks representing each of the representations and applying to encoded cardiac sound signals at the encoder output and feed the masked cardiac sound signals to the decoder 463. The masked cardiac sound signals may be inverted back to the denoised cardiac sound signals 403 using the decoder 463. In embodiments, the masks may be created by using a temporal convolutional network including stacked 1-D dilated convolutional blocks.

In embodiments, the separator 465 may include a depthwise convolution. The depthwise convolution may divide the input into individual channels based on the number of the vibration sensors 208, convolve each channel with an individual depthwise kernel with depth multiplier output channels, and concatenate the convolved results along the channels axis. The depthwise convolution may not mix data across the cardiac signals of each user. The separator 465 may further include a pointwise convolution. The pointwise convolution may use a 1×1 kernel. The 1×1 kernel may iterates through each cardiac signals of each user and audio input signals 103 of each vibration sensor 208, and thus maintain a depth equal to the number of input signal channels, such as the number of users multiplying the number of vibration sensors 208. The pointwise convolution may be used in conjunction with depthwise convolutions to produce depthwise-separable convolutions.

In some embodiments, the convolved outputs may be normalized and converted using an activation function for training and verification purposes, as described in detail further below. The activation function may be linear or nonlinear. The activation function may be a parametric rectified linear unit that learns the slope parameters of each channel at a layer of the neural network. The learned slope parameters are used to increase the accuracy of the model without any additional computational overhead.

In some embodiments, after delivering the feature data to the final layer of the neural network, a global layer normalization may be conducted to normalize both the channel and the time dimensions using cumulative layer normalization. The resulting normalized feature data may have the same number of channels as the cardiac sound data (the output of the encoder). The normalized feature data may further be operated through a second activation function to verify the generated masks. The second activation function may be linear or nonlinear. The second activation function may be a Softmax function or a Sigmoid function.

The decoder 463 may generate the denoised cardiac sound signals 403 for each user. The denoised cardiac sound signals 403 may be fed to the second neural network 442, where the weak alignment module 242 may convert the denoised cardiac sound signal 403 to the simulated ECG signals 307. The simulated ECG signals 307 may resemble measured ECG signals in the peak positions, especially the R peaks and T peaks, the relative waveform shapes. In some embodiments, the simulated ECG signals 307 have normalized amplitude with arbitrary units and time unit in seconds. In some embodiments, the weak alignment module 242 may compare with the simulated R peak locations, RR intervals, and the heart rate with the patterns of the ECG signals in the historical ECG data 247 (e.g. as illustrated in FIG. 2) and generate or modify the simulation ECG signals. In some embodiments, the second neural network 442 may be a part of the first neural network 432, either in conjunction or in parallel, such that the denoised cardiac sound signals 403 the isolated cardiac sound data into the time domain to generate the simulated ECG data based on the weak alignment between cardiac sounds and the ECG

FIG. 4B depicts the training of the neural networks 432 and 442 using measured cardiac audio samples 413 and measured ECG signals 305 associated with the measured cardiac audio samples 413. The first neural network 432 may be trained based on a dataset containing a wide range of the measured cardiac audio samples 413 of multiple users and audio input signal samples 411 of the multiple users recorded using multiple independent vibration sensors 208 (e.g. as illustrated in FIG. 1). The second neural network may be trained using the measured cardiac audio samples 413 and sample measured ECG signals 305. The audio input signal samples 411, the measured cardiac audio samples 413, and the sample measured ECG signals 305 are simultaneously recorded from the same sample users in the same environment.

In embodiments, the neural networks, such as the first neural network 432 or the second neural network 442, may be trained based on the activation functions. The encoder 461 may generate encoded cardiac sound data h=(Wx+b) that is transformed from the cardiac sound data 401 of several input channels. The encoded cardiac sound of one channel may be represented as hᵢⱼ=g(Wxᵢⱼ+b) from the raw cardiac sound data xᵢⱼ, which is then used to reconstruct output \tilde{x}ᵢⱼ=f(W^{T}hᵢⱼ+b′). The neural networks may reconstruct outputs, such as the masks as outputs of the separator 465, the denoised cardiac sound signals 403, or the simulated ESG signals 307, x′=(W^{T}h+b′), where W is weight, b is bias, W^{T} and b′ are transverse values of W and b and are learned through back propagation. In this operation, the neural network may calculate, for each input cardiac sound data 401, a distance between an input cardiac sound data x and a reconstructed cardiac sound data x′ or simulated ECG signal 307, to yield a distance vector |x−x′|. The calculated vectors may be a high or a low level dimensional dataset, such as 1D, 2D, or nD vectors. The neural networks may minimize the loss function which is a utility function as the sum of all distance vectors. The training process may enable the neural network to learn linear or non-linear representations of the input cardiac sound data. The accuracy of the predicted output such as the masks as outputs of the separator 465, the denoised cardiac sound signals 403, or the simulated ESG signals 307, may be evaluated by satisfying a preset value. For example, an accuracy and area under the curve (AUC) value may be computed using an output score from the activation function (e.g. the Softmax function or the Sigmoid function). For example, the system 100 may assign the preset value of the AUC with value of 0.7 to 0.8 as acceptable simulation, 0.8 to 0.9 is as excellent simulation, or more than 0.9 as outstanding simulation. After the training satisfying the preset value, the updated denoising machine learning algorithm and the updated weak alignment machine learning algorithm are stored in the denoising module 232 and the weak alignment module 242, respectively, which are used to process the cardiac sound data 401, as illustrated in FIG. 4A. In embodiments, the user may further provide measured cardiac sound data or measured ECG data to continue train the neural networks for individualization.

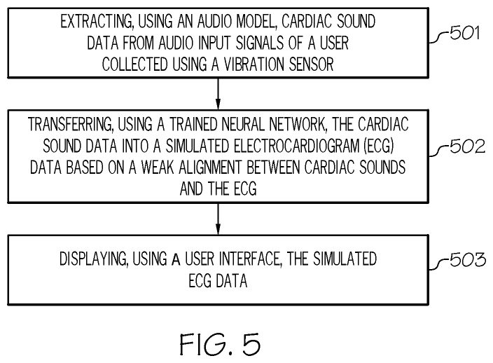

FIG. 5 illustrates a flow diagram of illustrative steps for generating simulated ECG data using the contactless cardiac monitoring system of the present disclosure. At block 501, the method for contactless monitoring of cardiac activities include extracting, using the audio model, cardiac sound data from audio input signals of the user collected using the vibration sensor. At block 502, the method for contactless monitoring of cardiac activities include transferring, using a trained neural network, the cardiac sound data into the simulated ECG data based on the weak alignment between cardiac sounds and ECG. At block 503, the method for contactless monitoring of cardiac activities include displaying, using a user interface, the simulated ECG data.

In embodiments, the vibration sensor may be selected from, without limitations, an audio sensor, an accelerometer sensor, or a combination thereof. The audio sensor may be, without limitations, an air-coupled audio sensor, a condenser microphone, an electret microphone, or a piezoelectric microphone. The accelerometer sensor may be embedded in a fixture.

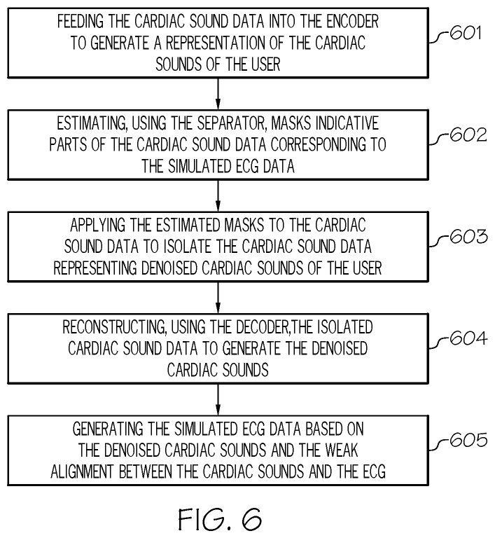

FIG. 6 illustrates a flow diagram of illustrative steps for generating simulated ECG data using a neural network of the contactless cardiac monitoring system of the present disclosure, according to one or more embodiments shown and described herein. At block 601, the method of transferring the cardiac sound data into the simulated ECG data using the neural network includes feeding the cardiac sound data into the encoder to generate a representation of the cardiac sounds of the user. At block 602, the method of transferring the cardiac sound data into the simulated ECG data using the neural network includes estimating masks indicative parts of the cardiac sound data corresponding to the simulated ECG data using the separator.

At block 603, the method of transferring the cardiac sound data into the simulated ECG data using the neural network includes applying the estimated masks to the cardiac sound data to isolate the cardiac sound data representing denoised cardiac sounds of the user. At block 604, the method of transferring the cardiac sound data into the simulated ECG data using the neural network includes reconstructing the isolated cardiac sound data to generate the denoised cardiac sounds using the decoder. At block 605, the method of transferring the cardiac sound data into the simulated ECG data using the neural network includes generating the simulated ECG data based on the denoised cardiac sounds and the weak alignment between the cardiac sounds and the ECG.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including matters of logic with respect to the arrangement of steps, operational flow, order of components, or orientation of components; plain meaning derived from grammatical organization or punctuation, and; the number or type of embodiments described in the specification.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

1. A system for contactless monitoring of cardiac activities, the system comprising:
a vibration sensor operable to collect audio input signals of a user; and
a processor operable to:
extract, using an audio model, cardiac sound data from the audio input signals,
transfer, using a trained neural network, the cardiac sound data into a simulated electrocardiogram ECG data based on a weak alignment between cardiac sounds and ECG.

2. The system of claim 1, wherein the vibration sensor is selected from an audio sensor, an accelerometer sensor, or a combination thereof.

3. The system of claim 2, wherein the audio sensor is an air-coupled audio sensor, a condenser microphone, an electret microphone, or a piezoelectric microphone.

4. The system of claim 2, wherein the accelerometer sensor is embedded in a fixture.

5. The system of claim 1, wherein the audio model extracts the cardiac sound data by filtering, normalizing, or segmenting the audio input signals.

6. The system of claim 5, wherein the system further comprises a signal processing filter to isolate frequencies of the audio input signals between 25 Hz and 50 Hz.

7. The system of claim 5, wherein the normalization comprises adjusting amplitudes of the audio input signals by peak amplitude normalization, root mean square normalization, or loudness normalization.

8. The system of claim 1, wherein the neural network comprises an encoder, a separator, and a decoder, wherein the neural network is operable to:
feed the cardiac sound data into the encoder to generate a representation of the cardiac sounds of the user,
estimate, using the separator, masks indicative parts of the cardiac sound data corresponding to the simulated ECG data,
apply the estimated masks to the cardiac sound data to isolate the cardiac sound data representing denoised cardiac sounds of the user,
reconstruct, using the decoder, the isolated cardiac sound data to generate the denoised cardiac sounds, and
generate the simulated ECG data based on the denoised cardiac sounds and the weak alignment between the cardiac sounds and the ECG.

9. The system of claim 1, wherein the weak alignment between the cardiac sounds and the ECG is established based on correlated pair features between the cardiac sounds and the ECG.

10. The system of claim 9, wherein the correlated pair features comprise R peaks or T peaks in the ECG and S1 features or S2 features in the cardiac sounds.

11. The system of claim 1, wherein the neural network outputs the simulated ECG data based on estimated R peak locations, RR intervals, and heart rates.

12. The system of claim 1, wherein the neural network is trained using sample cardiac sound signals and sample ECG signals, wherein the sample cardiac sound signals and the sample ECG signals are simultaneously recorded from same sample users.

13. A method for contactless monitoring of cardiac activities comprising:
extracting, using an audio model, cardiac sound data from audio input signals of a user collected using a vibration sensor; and
transferring, using a trained neural network, the cardiac sound data into a simulated electrocardiogram (ECG) data based on a weak alignment between cardiac sounds and ECG.

14. The method of claim 13, wherein the vibration sensor is selected from an audio sensor, an accelerometer sensor, or a combination thereof.

15. The method of claim 14, wherein:
the audio sensor is an air-coupled audio sensor, a condenser microphone, an electret microphone, or a piezoelectric microphone; and
the accelerometer sensor is embedded in a fixture.

16. The method of claim 13, wherein:
the audio model extracts the cardiac sound data by filtering, normalizing, or segmenting the audio input signals;
the filtering comprises isolating frequencies of the audio input signals between 25 Hz and 50 Hz; and
the normalization comprises adjusting amplitudes of the audio input signals by peak amplitude normalization, root mean square normalization, or loudness normalization.

17. The method of claim 13, wherein the neural network comprises an encoder, a separator, and a decoder, wherein the neural network is operable to:
feed the cardiac sound data into the encoder to generate a representation of the cardiac sounds of the user,
estimate, using the separator, masks indicative parts of the cardiac sound data corresponding to the simulated ECG data,
apply the estimated masks to the cardiac sound data to isolate the cardiac sound data representing denoised cardiac sounds of the user,
reconstruct, using the decoder, the isolated cardiac sound data to generate the denoised cardiac sounds, and
generate the simulated ECG data based on the denoised cardiac sounds and the weak alignment between the cardiac sounds and the ECG.

18. The method of claim 13, wherein the weak alignment between the cardiac sounds and the ECG is established based on correlated pair features between the cardiac sounds and the ECG, and the correlated pair features comprise R peaks or T peaks in the ECG and S1 features or S2 features in the cardiac sounds.

19. The method of claim 13, wherein the neural network outputs the simulated ECG data based on estimated R peak locations, RR intervals, and heart rates.

20. The method of claim 13, wherein the neural network is trained using sample cardiac sound signals and sample ECG signals, wherein the sample cardiac sound signals and the sample ECG signals are simultaneously recorded from same sample users.
